(19) United States
(12) Patent Application Publication
(10) Pub. No.: US 2024/0409901 A1
(43) Pub. Date: Dec. 12, 2024

(54) METHODS FOR DEVELOPING A CELL LINE FOR PRODUCING VIRUS IN SUSPENSION CELL CULTURE

(71) Applicant: PORTON ADVANCED SOLUTIONS LTD., Suzhou, Jiangsu (CN)

(72) Inventors: Dichao HU, Suzhou, Jiangsu (CN); Jun HUA, Suzhou, Jiangsu (CN); Weimeng ZHANG, Suzhou, Jiangsu (CN); Fei TENG, Suzhou, Jiangsu (CN); Shuhui DING, Suzhou, Jiangsu (CN)

(21) Appl. No.: 18/700,268

(22) PCT Filed: Oct. 27, 2022
(86) PCT No.: PCT/CN2022/127878

§ 371 Date: Apr. 10, 2024

(30) Foreign Application Priority Data
Oct. 27, 2021 (CN) 202111258035.1
Sep. 30, 2022 (CN) 202211213168.1

(51) Int. Cl. C12N 5/071; C12N 15/86; C12Q 1/6897

(52) U.S. Cl. CPC C12N 5/0686; C12N 15/86; C12Q 1/6897; C12N 2510/04; C12N 2740/15043; C12N 2740/15051; C12N 2750/14143; C12N 2750/14151; C12Q 2600/158

(57) ABSTRACT

A method for generating a cell line for producing virus in suspension cell culture. The method comprises generating a plurality of cell clones growing in adherent cell culture, identifying a group of cell clones with high transfection efficiency, generating a plurality of cell clones growing in suspension cell culture and identifying a cell clone suitable for producing virus in suspension cell culture. The method disclosed herein the method can shorten the domestication time from at least 1 month to 7-14 days, and the amount of medium used during the domestication process can also be greatly reduced, reducing production costs.

### CROSS-REFERENCE TO RELATED APPLICATIONS

This application claims the priorities of Chinese application CN202111258035.1, filed Oct. 27, 2021, and Chinese application CN202211213168.1, filed Oct. 1, 2022, the entire disclosure of which are incorporated herein by reference.

### FIELD OF THE INVENTION

The present invention generally relates to biotechnology. More particularly, the invention relates to a method for developing a cell line for producing virus in suspension cell culture.

### BACKGROUND OF THE INVENTION

Gene therapy has become a promising approach to treat various diseases. To deliver the transgene into target cells, viral vectors, such as adenovirus vectors (ADVs), adeno-associated virus vectors (AAVs) and lentivirus vectors (LVVs) are often used. More recently, Chimeric Antigen Receptor T-Cell (CAR-T) immunotherapy has been successfully developed to treat tumors. LVVs are usually used to deliver CAR gene into the T cells due to the advantages of high transduction efficiency, integration of T cell genome and continuous expression of target proteins. Besides, the viral vectors used for gene therapy are usually adeno-associated virus vectors (AAV) or adenovirus vectors (ADV). Therefore, the production of viral vectors have become a key step in gene therapy and CAR-T therapy.

At present, most processes for virus production are based on fetal bovine serum (FBS)-dependent adherent cell technology (such as HEK293T), which have the advantages of high lentivirus expression titer and less hardware investment. However, the adherent cell technology relies on FBS, which is incompatible with current regulations, and necessitates rigorous audits of FBS manufacturers, as well as evaluation and testing for removal. In addition, differences between batches and origins of FBS may also lead to batch-to-batch variation in the expressed lentivirus. Another key issue to be addressed is that most of the adherent cell technologies currently used are cell factories, making it difficult to scale up. At present, cell factory-based lentivirus production is usually 10-layer cell factory (CF-10) or 40-layer cell factory (CF-40), usually 10 to 20, which poses great challenges for operation and manpower.

Suspension cells can be grown at high densities in chemically defined media (CD media) independent of FBS, and can be easily scaled up into bioreactors using cell culture shake flasks (e.g., 2 L, 15 L, 50 L, 200 L, 2000 L) for production, so it is easy to scale up.

Some technologies for suspension domestication of HEK293T adherent cells have been developed, which adopts the scheme of gradually reducing the amount of FBS in the culture medium, so that HEK293T adherent cells are gradually domesticated to a condition that does not require FBS. The suspension domestication scheme of FBS medium has the disadvantage of long time-consuming. Usually, the domestication time needs at least one month, and the titer of lentivirus produced by the obtained domesticated suspension HEK293T cells after plasmid packaging is low, and the HCP content is relatively high, which makes the subsequent purification process very difficult.

For suspension domestication of HEK293 cells, the traditional strategy is to gradually reduce and replace DMEM or EMEM and fetal bovine serum, that is, the serum concentration is gradually reduced from 10% to 7.5%, 5%, 2.5%, 1%, and DMEM or EMEM is also gradually replaced by suspension culture medium without serum. However, this suspension domestication process is time-costing, usually takes 3-5 months to complete. Furthermore, as a sub triploid cell of human embryonic kidney, HEK293 is a cell with strong chromosome heterogeneity, which leads to slow growth rate and low transfection efficiency of HEK293 protocells, resulting in low unit yield of viral vector titer. Therefore, there is a continuing need to develop cell lines for producing virus in suspension cell culture.

### BRIEF SUMMARY OF THE INVENTION

In one aspect, the present disclosure provides a method for developing a cell line for suspension cell culture. In some embodiments, the method comprises: (i) generating a plurality of cell clones growing in adherent cell culture, each cell clone comprising a group of cells sharing a common ancestry cell; (ii) identifying a group of cell clones with high transfection efficiency; (iii) generating a plurality of cell clones growing in suspension cell culture; and (iv) selecting a cell clone suitable for producing virus in suspension cell culture.

In one embodiment, the method for selecting a cell for suspension cell culture comprises: (i) generating a plurality of cell clones growing in adherent cell culture, each cell clone comprising a group of cells sharing a common ancestry cell; (ii) identifying a group of cell clones with high transfection efficiency by: allocating a cell fraction from each cell clones, respectively; transfecting the cell fraction from each cell clones, respectively, with a report plasmid comprising a first reporter gene; and identifying a group of cell clones with high transfection efficiency from the plurality of cell clones according to the expression of the first reporter gene in the cell fraction from each cell clones; (iii) generating a plurality of cell clones growing in suspension cell culture by: allocating a cell fraction from each of the group of cell clones with high transfection efficiency, respectively; culturing the cell fraction from each of the group of cell clones with high transfection efficiency, respectively, in a medium that allows the cell fraction from each of the group of cell clones with high transfection efficiency to grow in suspension cell culture, thereby generating a plurality of cell clones growing in suspension cell culture; and (iv) selecting a cell clone suitable for producing virus in suspension cell culture by: allocating a cell fraction from each of the plurality of cell clones growing in suspension cell culture, respectively; transfecting the cell fraction from each of the plurality of cell clones growing in suspension cell culture, respectively, with (a) a virus transfer plasmid comprising a second reporter gene and (b) a virus packaging plasmid; collecting virus produced by the cell fraction from each of the plurality of cell clones growing in suspension cell culture, respectively; transducing a cell with the virus produced by the cell fraction from each of the plurality of cell clones growing in suspension cell culture, respectively; and identifying a cell clone producing high titers of virus from the plurality of cell clones growing in suspension cell culture according to the expression of the second reporter gene in the cell, thereby generating a cell line suitable for producing virus in suspension cell culture.

In some embodiments, the cell for suspension cell culture is selected from the group consisting of HEK 293T, HEK293F, HEK293, 293S, BHK, BHK-21, CHO, CHO/dhfr-, or CHO K1 cell. In some embodiments, the cell for suspension cell culture is a HEK293T cell.

In some embodiments, the first reporter gene and/or the second reporter gene encode(s) a fluorescent protein. In some embodiments, the fluorescent protein is GFP, such as EGFP, or other colors of fluorescent proteins, such as EYFP, EBFP, etc.

In some embodiments, in step (ii), the cell fraction from each cell clones is transfected after logarithmic growth phase.

In some embodiments, step (ii) further comprises screening cell clones producing high tier virus in adherent cell culture from the cell clones with high transfection efficiency.

In some embodiments, in step (iii), the medium is a serum free medium. In some embodiments, the serum free medium contains VP-SFM, SFM4 HEK293, SFM4 Transfx293, HEK293 MaxX, OPM 293 CD05, OptiVitro 293, HEK293-04 Prototype, HEK293-13 Prototype, HEK Vip NX, HEK Vip NB, HEK TF or HEK GM medium. In some embodiments, the serum free medium further contains 2-8 mM L-alanyl-L-glutamine dipeptide (GlutaMax™). In some embodiments, the serum free medium further comprises 0.05-1% (v/v) Anti Clumping Agent. In certain embodiments, for example, when the cell for suspension cell culture is a HEK293T cell, the serum free medium contains VP-SFM medium and 2 mM GlutaMax™. In certain embodiments, for example, when the cell for suspension cell culture is HEK 293, the serum free medium contains (a) SFM4 HEK293 medium or (b) OPM 293 CD05 medium, 2-8 mM GlutaMax™ and 0.05-1% (v/v) Anti Clumping Agent. Optionally, the concentration of GlutaMax™ in the serum free medium is selected from the group consisting of 2 mM, 4 mM, 6 mM and 8 mM. Optionally, the concentration of Anti Clumping Agent in the serum free medium is selected from the group consisting of 0.05%, 0.1% (v/v), 0.2% (v/v), 0.5% (v/v) and 1% (v/v). In certain embodiments, the serum free medium contains SFM4 HEK293 serum, 3.5-4.5 mM GlutaMax™ and 0.1-0.5% (v/v) Anti Clumping Agent. In one embodiment, the serum free medium contains SFM4 HEK293 medium, 4 mM GlutaMax™ and 0.1% (v/v) Anti Clumping Agent. It can be understood that in some embodiments, the Anti Clumping Agent in the serum free medium can be omitted.

In some embodiments, the cell fraction from each of the group of cell clones is cultured in an expansion culture medium before being culture in the serum free medium. In some embodiments, the expansion culture medium contains (a) VP-SFM medium, or (b) Pro293 medium and 0%-2% (v/v) FBS. In some embodiments, the expansion culture medium further contains 2-8 mM GlutaMax™. Optionally, the concentration of FBS in the expansion culture medium is selected from the group consisting of 0% (v/v), 0.5% (v/v), 1% (v/v), and 2% (v/v). In some embodiments, the concentration of GlutaMax™ in the expansion culture medium is selected from the group consisting of 2.0 mM, 4 mM, 6 mM and 8 mM. In some embodiments, the expansion culture medium contains (a) VP-SFM medium or (b) Pro293 medium, 0.5%-1.5% (v/v) FBS, and 3-5 mM GlutaMax™. In one embodiment, the expansion culture medium contains VP-SFM medium and 1% (v/v) FBS. In another embodiment, the expansion culture medium contains VP-SFM medium, 1% (v/v) FBS and 4 mM GlutaMax™.

In some embodiments, the cell fraction from each of the group of cell clones is cultured in an adaptive subculture medium after being cultured in the serum free medium. In some embodiments, the cell fraction from each of the group of cell clones is cultured in the adaptive subculture until cell viability is 93-98%. In some embodiments, the adaptive subculture medium is Transpro CD01 medium containing 4 mM GlutaMax™ and 0.1% Anti-clumping agent. In some embodiments, the cell fraction from each of the group of cell clones is cultured in the adaptive subculture medium when the cell fraction has a density of 5-8E+05 cells/mL.

In some embodiments, in step (iii), the cell fraction from each of the group of cell clones is cultured in an adaptive subculture medium after being cultured in the serum free medium. In some embodiments, the cell fraction from each of the group of cell clones is cultured in the adaptive subculture until cell viability is 93-98%. In some embodiments, the adaptive subculture medium is Transpro CD01 medium containing 4 mM GlutaMax™ and 0.1% Anti-clumping agent. In some embodiments, the cell fraction from each of the group of cell clones is cultured in the adaptive subculture medium when the cell fraction has a density of 5-8E+05 cells/mL.

In some embodiments, the expression of the second reporter gene is measured via flow cytometry and fluorescence microscope.

In some embodiments, the cell fraction of step (ii) or (iv) is transfected using PEIpro, PEI MAX or VirusGen.

In some embodiments, the report plasmid has a concentration of 0.1-0.6 g/cm². In some embodiments, the report plasmid has a concentration of 0.5-2 μg/cm².

In some embodiments, the virus transfer plasmid and/or the second reporter gene has a concentration of 1-5 μg/mL. In some embodiments, the virus transfer plasmid and/or the second reporter gene has a concentration of 1-4 μg/mL.

In some embodiments, the plurality of cell clones in step (i) is cultured in medium containing (a) VP-SFM or (b) Pro293 medium, 2-8 mM GlutaMax™ and 2-10% (v/v) FBS. In some embodiments, the plurality of cell clones in step (i) is cultured in medium containing (a) VP-SFM or (b) Pro293 medium, 2-8 mM GlutaMax™ and 5-10% (v/v) FBS. Optionally, the concentration of GlutaMax™ in the medium is selected from the group consisting of 2.0 mM, 4 mM, 6 mM and 8 mM. Optionally, the concentration of FBS in the expansion culture medium is selected from the group consisting of 2% (v/v), 4% (v/v), 5% (v/v), and 8% (v/v). In some embodiments, the plurality of cell clones in step (i) is cultured in medium containing VP-SFM medium, 3.5-4.5 mM GlutaMax™ and 2-10% (v/v) FBS. In some embodiments, the plurality of cell clones in step (i) is cultured in medium containing VP-SFM or Pro293 medium, 3.5-4.5 mM GlutaMax™ and 10% (v/v) FBS. In one embodiment, the plurality of cell clones in step (i) is cultured in medium containing (a) VP-SFM or (b) Pro293 medium, 4 mM GlutaMax™ and 10% (v/v) FBS.

In some embodiments, the cell fraction from each of the group of cell clones is cultured in an expansion culture medium before being culture in the serum free medium. In some embodiments, the expansion culture medium contains (a) VP-SFM medium, or (b) Pro293 medium and 0%-2% (v/v) FBS. In some embodiments, the expansion culture medium further contains 2-8 mM GlutaMax™. In some embodiments, the expansion culture medium contains (a) VP-SFM medium or (b) Pro293 medium, 0.5%-1.5% (v/v) FBS, and 3-5 mM GlutaMax™. In one embodiment, the expansion culture medium contains VP-SFM medium and 1% (v/v) FBS. In another embodiment, the expansion culture medium contains VP-SFM medium, 1% (v/v) FBS and 4 mM GlutaMax™.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description of specific embodiments presented herein.

FIG. 1 illustrates an exemplary method of this invention.

FIG. 2A illustrates a fluorescent image of cloned cells with high transfection efficiency after transfection with lentiviral transfer plasmid in step A4.

FIG. 2B illustrates a fluorescent image of cloned cells with low transfection efficiency after transfection with lentiviral transfer plasmid in step A4.

FIG. 3A illustrates a fluorescence image reflects infection efficiency of EGFP lentivirus on cells producing high titers of virus after EGFP lentivirus packaging in step A5.

FIG. 3B illustrates a fluorescence image reflects infection efficiency EGFP lentivirus on cells producing low titers of virus after EGFP lentivirus packaging in step A5.

FIG. 4A illustrates a fluorescence image after the cells were transfected with EGFP transfer plasmids following resuspension and subculture in Dynamis medium in step B2.

FIG. 4B illustrates a fluorescence image after the cells were transfected with EGFP transfer plasmids following resuspension and subculture in LV-MAX medium in step B2.

FIG. 4C illustrates a fluorescence image after the cells were transfected with EGFP transfer plasmids following resuspension and subculture in SFM4HEK293 medium in step B2.

FIG. 4D illustrates a fluorescence image after the cells were transfected with EGFP transfer plasmids following resuspension and subculture in Transpro CD01 medium in step B2.

FIG. 5 illustrates a graph of the positive rate data of CAR in T cells activated by lentiviral transduction prepared using the packaging conditions of item 1 in the application example.

FIG. 6 illustrates a graph of the positive rate data of CAR in T cells activated by lentiviral transduction prepared using the packaging conditions of item 2 in the application example.

FIG. 7 illustrates the transfection efficiency of 03G11 at the stage of selecting adherent clones in Example 1.

FIG. 8 illustrates the transfection efficiency of 06D08 at the stage of selecting adherent clones in Example 1.

FIG. 9 illustrates the transfection efficiency of 19C03 at the stage of selecting adherent clones in Example 1.

FIG. 10 illustrates the transfection efficiency of 01F10 at the stage of selecting adherent clones in Example 1.

FIG. 11 illustrates the transfection efficiency of 12D06 at the stage of selecting adherent clones in Example 1.

FIG. 12 illustrates the transfection results of cells numbered 19C03 transfected with CD05 medium, PEIpro and VirusGEN as transfectants respectively at the stage of selecting domesticated clone cells in suspension in Example 1.

FIG. 13 the transfection results of cells numbered 19C03 transfected with PEIpro as transfectant, LV-EGFP as transfer plasmid, Max X and CD05 as culture medium respectively at the stage of selecting domesticated clone cells in suspension in Example 1.

FIG. 14 the results of transfection of cells numbered 19C03 with VirusGEN as transfectant, LV-EGFP as transfer plasmid, Max X and CD05 as culture medium respectively at the stage of selecting domesticated clone cells in suspension in Example 1.

FIG. 15 illustrates the growth curve of 19C03 at the stage of selecting domesticated clone cells in suspension in Example 1

FIG. 16 illustrates the transfection efficiency of HEK293 adherent cells in Example 2.

### DETAILED DESCRIPTION OF THE INVENTION

In the Summary of the Invention above and in the Detailed Description of the Invention, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

Where a range of value is provided, it is understood that each intervening value, to the tenth of the unit of the lower limit unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, the embodiments described herein can be practiced without there specific details. In other instances, methods, procedures and components have not been described in detail so as not to obscure the related relevant function being described. Also, the description is not to be considered as limiting the scope of the implementations described herein. It will be understood that descriptions and characterizations of the embodiments set forth in this disclosure are not to be considered as mutually exclusive, unless otherwise noted.

### Definition

The following definitions are used in the disclosure:

It is understood that as used herein and in the appended claims, the singular forms “a,” “an,” and “the” include the plural reference unless the context clearly dictates otherwise. Thus, for example, a reference to a “cell” is a reference to one or more detection antibodies, and includes equivalents thereof known to those skilled in the art and so forth.

The term “comprise” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components.

The term “lentiviruses” include the members of the bovine lentivirus group, equine lentivirus group, feline lentivirus group, ovinecaprine lentivirus group and primate lentivirus group. Examples of lentiviruses suitable for the methods and use of the invention include, but are not limited to, HIV and their pseudotypes such as HIV-1, HIV-2, HIV-1/HIV-2 pseudotype, HIV-1/SIV, FIV, caprine arthritis encephalitis virus (CAEV), equine infectious anemia virus, bovine immunodeficiency virus and Vesucular Stomatitis Virus G-pseudotyped lentivirus (VSVG pseudotypede).

Lentiviruses are enveloped viruses, and are significantly different in terms of virus structure and life cycle from other viruses used for delivery of nucleic acid into cells, such as adeno-associated viruses (AAV). Lentiviruses are composed of 2 copies of RNA, a nuclear capsid (NC), a Capsid (CA) a membrane associated matrix (MA), envelope proteins such as surface glycoproteins and transmembrane proteins and enzymes such as integrase (IN), protease (PR), reverse transcriptase (RT) and accessory proteins (e.g., Nef, Vif, Vpu, Vpr). Lentiviruses infect cells by binding of a surface glycoprotein of the virus to a receptor on the cell. The membranes of the envelope of the virus and the cell then fuse allowing the virus to enter the cell. Following entry, uncoating of viral RNA and reverse transcription takes place which leads to the formation of a pre-integration complex, which contains double stranded DNA, RT, IN, Vpr (or Vpx in HIV-2) NC, and some copies of the MA (Suzuki and Craigie 2007, Depienne et al., 2000, Bukrinsky et al., 1993 and Miller et al., 1997). Once the provirus enters the nuclear envelope, the viral DNA integrates within the cell genome. Normal cellular functions of transcription and translation are followed by assembly of structural viral proteins with viral RNA and subsequent viral budding.

Lentiviruses are desirable for delivery of nucleic acid into cells in part because they can infect non-dividing cells by actively entering the nucleus through the nuclear envelope. By contrast, other retroviruses require cell division for infection due to the fact that it cannot enter the nuclear envelope of a non-dividing cell.

By “vector” is meant a genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

The term “transfection” is used to refer to the uptake of foreign nucleic acid (e.g., DNA) by a cell, and a cell has been “transfected” when exogenous nucleic acid (e.g., DNA) has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties into suitable host cells.

By “well-growing” is meant the cells or cell clones at logarithmic growth phase and have a cell viability rate of more than 90%.

The term “cell line” refers to population of cells generated by primary cell culture after successful passage for the first time. A cell line can be maintained in culture for an extended period of time, retaining stability of certain phenotypes and functions.

### Methods for Developing a Cell Line for Producing Virus in Suspension Cell Culture

The present disclosure in one aspect provides a method for selecting a cell for suspension cell culture. The cell selected can be used for generating virus (e.g., lentivirus, AAV) that is used in transgene therapy, such as chimeric antigen receptor-T cell therapy.

In some embodiments, the method comprises: (i) generating a plurality of cell clones growing in adherent cell culture, each cell clone comprising a group of cells sharing a common ancestry cell; (ii) identifying a group of cell clones with high transfection efficiency; (iii) generating a plurality of cell clones growing in suspension cell culture; (iv) identifying a cell clone suitable for suspension cell culture.

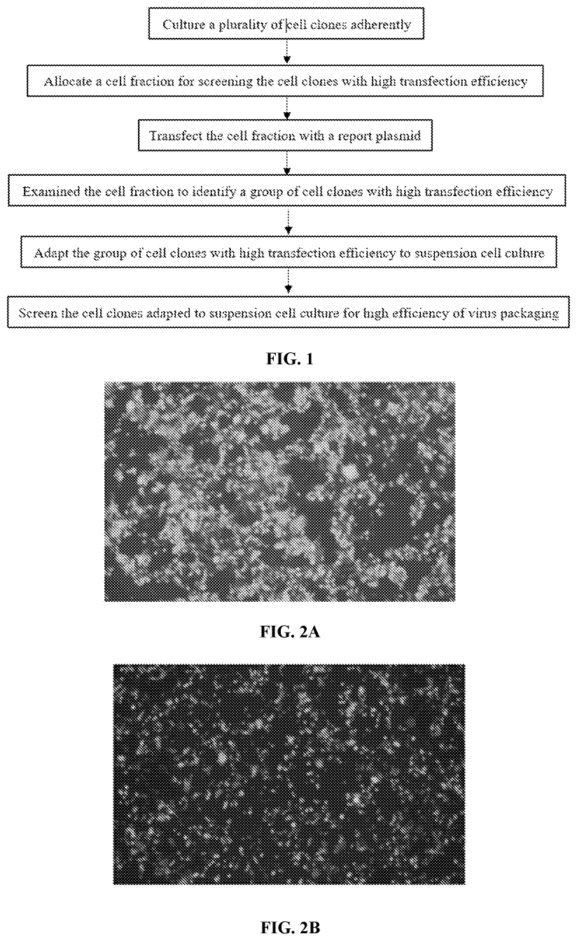

An exemplary embodiment of the method for selecting a cell for suspension cell culture described herein is illustrated in FIG. 1. Referring to FIG. 1, in the initial step, a plurality of cell clones are cultured adherently with a complete medium. As used herein, a cell clone means a group of cells sharing a common ancestry cell, i.e., proliferated from a single ancestry cell. In one embodiment, to prepare a plurality of cell clones, a population of cells are separated or sorted into different cell culture wells so that each well contains only one single cell. Each sorted single cell is then cultured and grown into a cell clone. In certain embodiments, at least 100, 150, 200, 250, 300, 350, 400, 500, 600, 700, 800, 900, 1000, 1200, 1400, 1600, 1800, or 2000 cell clones are cultured adherently in the initial step.

Cells that can be used to generate the cell clones include HEK 293T, HEK293F, HEK293, 293S, BHK, BHK-21, CHO, CHO/dhfr-, or CHO K1 cell. In some embodiments, the cell clones are generated from HEK293T cells. In some embodiments, the cell clones are generated from HEK293 (for example, ATCC No. CRL1573).

The medium and condition used to culture the cell clones are selected according to the type of cells generating the cell clones and are known in the art. Typically, the medium used for cell culture are made from commercially available serum free base medium, with other components and supplements added depending on the cell types and the purposes of cell culture. Non-limiting exemplary serum free medium include FreeStyle™293 (Gibco®, Life Technologies), DMEM/F12 (Gibco®, Life Technologies), SFM4Transfx-293 (HyClone™, ThermoScientific), CDM4HEK293 (HyClone™, ThermoScientific), StemPro-34SFM (Gibco®, Life Technologies), FreeStyle F17 (Gibco®, Life Technologies), 293SFM II (Gibco®, Life Technologies), CD293 (Gibco®, Life Technologies) medium, VP-SFM, SFM4 HEK293, HEK293 MaxX, OPM 293 CD05, OptiVitro 293, HEK293-04 Prototype, HEK293-13 Prototype, HEK Vip NX, HEK Vip NB, HEK TF and HEK GM.

Supplements of cell culture include, for example, serum, hormones, growth factors, antibiotics, amino acids (e.g., L-glutamine), carbohydrates (e.g., glucose), vitamins, etc. Typical medium for adherent cell culture contain 10% fetal bovine serum (FBS). In some embodiments, the complete medium used for adherent cell culture is VP-SFM medium comprising 2-8 mM GlutaMax™ and 10% FBS.

In certain embodiments, before proceeding to the next step, the cell clones are cultured until reaching the logarithmic growth phase. At the logarithmic growth phase, the cells actively proliferate and increase exponentially in cell density.

Referring to FIG. 1, in the next step, a cell fraction is allocated from each cell clone for screening the cell clones with high transfection efficiency while the rest of the cells in each cell clones are cultured or stored for further screening steps. In certain embodiments, the cell fraction from each cell clone is transfected with a report plasmid. A report plasmid, also known as a reporter plasmid, comprises a reporter gene encoding a detectable protein or enzyme. Usually, the report plasmid is used to indicate transcription or expression level of a target protein or indicate transfection efficiency of cells through expression of the reporter gene. In certain embodiments, use of the reporter plasmid aims to indicate the transfection efficiency of cells through expression of the reporter gene encoding a fluorescent protein, such as GFP. In certain embodiment, the report plasmid is generated based on a viral vector. The cell fraction from each cell clone is then examined to identify a group of cell clones with high transfection efficiency. In some embodiments, the cell clones having high transfection efficiency are identified according to the expression of the report gene in the cell fractions. In certain embodiments, at least 10, 15, 20, 25, 30, 40, 50, 60, 70, 80, 90, 100 cell clones are identified as having high transfection efficiency.

The group of cell clones that demonstrate high transfection efficiency are then cultured to adapt to suspension cell culture. In common practice, adherent cultured cells can adapt to suspension cell culture by gradually decreasing the amount of serum (e.g., FBS) in the culture medium. In certain embodiments of the present disclosure, the method disclosed herein adopts a quicker approach to domesticate the adherent cell clones to suspension cell culture. In one embodiment, the cell clones to be domesticated are cultured with a medium comprising 1% FBS (e.g., VP-SFM medium comprising 2 mM GlutaMax™ and 1% FBS), and then subjected to expansion culture with FBS free medium (e.g., is VP-SFM medium comprising 2 mM GlutaMax™). The cell clones are then subject to adaptive subculture with FBS free medium successively. Thereafter, cell clones with a survival rate of at least 90% (e.g., 93-98%), in good dispersion and no obvious aggregation are selected. In certain embodiments, at least 5, 10, 15, 20, or 25 cell clones are adapted to suspension cell culture.

In the next step, the cell clones adapted to suspension cell culture are screened for high efficiency of virus packaging. A cell fraction is allocated from each cell clone for screening while the rest of the cells in each cell clones are cultured or stored for future use. In certain embodiments, to conduct the screening, the cell fraction from each cell clone is transfected with a virus transfer plasmid comprising a reporter gene and a virus packaging plasmid. A packaging plasmid refers to a plasmid system or mix, which is used together with a viral vector to package virus. Different viral vectors rely on different packaging plasmids. Usually, a packaging plasmid comprises basic components such as Gag, Pol, Rev and VSV-G, etc. In some embodiments, the basic components are comprised in different plasmids, which constitutes a packaging plasmid system. To test whether the cell clone is able to produce high titers of virus, virus is collected from culture supernatant in each cell fraction and used to transduce cells, e.g., HEK293T cells, from which a cell clone producing high titers of virus is identified according to the reporter gene in the cells transduced with the virus. The cell clone producing high titers of virus is selected as a cell line suitable for producing virus in suspension cell culture.

In one embodiment, the method disclosed herein is for developing an HEK293T cell line for producing lentivirus in suspension cell culture. The method comprises following steps:

S1. Screening cell clones suitable for lentivirus packaging from HEK293T cells growing in adherent cell culture, which specifically includes the following sub-steps:

A1. Resuscitating the HEK293T cells, and culturing the cells adherently using DMEM medium containing 10% FBS.

A2. Culturing HEK293T cells adherently in logarithmic growth phase using VP-SFM culture medium complemented with 2 mM GlutMax and 10% FB after the cells in step A1 were in well-growing state.

A3. Plating the HEK293T cells in logarithmic growth phase in step A2 clonally (i.e., substantially one cell in each well) into 96-well plates, then adding VP-SFM medium complemented with 2 mM GlutMax and 10% FBS to each well.

A4. Carrying out clone observation after culturing for 5-7 days, and exchanging part of complete VP-SFM medium and 10% FBS. Thereafter, the culture is continued, until the cell clones are expanded to the required amount. Then the required amount of cell clone is digested with trypsin. A cell fraction from each cell clone is allocated for screening and the rest of the cell clones is cultured or stored as seed cells. To conduct the screening, the fraction of each cell clone is transfected with a lentivirus transfer plasmid comprising EGFP gene. The cell clones with high expression of EGFP are identified as having high transfection efficiency, and the seed cells of these cell clones are expanded to build a bank. VP-SFM medium complemented with 1% FBS is used during amplification culture.

S2. Conducting suspension domestication on the identified HEK293T clones having high transfection efficiency, specifically including the following sub-steps:

B1. Resuscitating the cell clones with high transfection efficiency using VP-SFM medium complemented with 2 mM GlutMax and 1% FBS, consuming the clones after 2-3 days of static culture. Then static culture is expanded using VP-SFM medium complemented with 2 mM GlutMax. The resulted cells are then collected for determination of viable cell density.

B2. Conducting oscillatory culture using Transpro CD02 medium complemented with 4 mM GlutMax and 0.1% Anti-clumping agent in shake flasks. Thereafter cells are resuspended, followed by further oscillatory culture in shaker. Adaptive subculture is then carried out until the cell viability rate reaches 93-98%, and the cells show good dispersion and no obvious aggregation, which are considered as domesticated HEK293T cell clones. Some of the domesticated HEK293T cell clones are allocated, expanded, and cryopreserved for use.

S3. Screening cell clones producing high titers of lentivirus, specific sub-steps thereof are as follows: resuscitating the frozen HEK293T cell clones obtained in sub-step B2 using Transpro CD02 medium complemented with 4 mM GlutMax and 0.1% Anti-clumping agent, then transfecting with a lentivirus transfer plasmid comprising EGFP gene and a virus packaging plasmid. Then the supernatant containing EGFP lentivirus from each cell clone is harvested at 48 hours post transfection, followed by centrifugation at 1500 g for 15 minutes to remove cells and fragments thereof. The supernatant is then diluted at different times, and used for transduce adherent HEK293T cells in 96-well plates, then identifying cell clones producing high titers of virus by measuring supernatant with flow cytometry and fluorescence microscope.

Further effort was made to improve vector specific productivity. Parameters, such as cell density at transfection, total DNA amount used for transfection, methods to prepare DNA/PEI complex, vector harvest time, were evaluated. For example, in sub-step A4, the transfection can be carried out with the lentivirus transfer plasmid comprising EGFP gene and the lentivirus packaging plasmid after culturing for 24 hours. In some embodiments, the plasmid has a concentration of 0.1-0.6 μg/cm², such as 0.4 μg/cm². In some embodiments, the V/W ratio of transfection reagent PEIpro to plasmid was 1-3:1, such as 2:1.

In some embodiments, the step S2 further comprises sub-step B3, which is used to screen the domesticated cell clones with highest infection efficiency. Specifically, in sub-step B3, the cell clones obtained in B2 is further subjected to package with lentivirus including EGFP gene. The supernatant of lentivirus comprising EGFP gene is harvested at 48 h post transfection and is used to transduce HEK293T cells plated at different dilution times. After 48-72 hours, the infected HEK293T cells are collected, and the infection efficiency is detected by fluorescence microscope and FACS, so as to determine the cloned cells with highest infection efficiency. In some embodiments, when lentiviral package is carried out, the cells have a density of 2E+06 cells/mL, and the plasmid has a concentration of 1-3 μg/mL, for example, the four plasmids (virus transfer plasmid comprising EGFP gene, Pol/gag, VSV-g and Rev) have a concentration of 2 μg/mL, the DNA molar ratio of four plasmids is 2:1:1:1, the V/W ratio of transfection reagent PEIpro to plasmid DNA is 1-3:1, preferably 2:1. In some embodiments, the supernatant of lentivirus comprising EGFP is harvested at 48 h post transfection and is used to infect the HEK293T plated at different dilution times.

In some embodiments, the method disclosed herein is for developing an HEK293 cell line for producing adeno-associated virus (AAV), adenovirus (ADV) or oncolytic virus (OV) in suspension cell culture. The method comprises following steps:

S1. Generating cell clones from HEK293 cells growing in adherent cell culture with limiting dilution assay to obtain about 180-250 cell clones, which specifically includes the following operations:

Plating HEK293 cells adherently in a density of 1 cell/well in 900-2000 wells and conducting preliminary culture to obtain 100-250 well-growing cell clones. During the preliminary culture, the cells are cultured for 6-8 days and then half of the medium is exchanged with unused fresh medium, with an exchange frequency of 3-4 days each time in following culture.

In some embodiments, the number of cell clones obtained in step S1 is 190-230. In one embodiment, the number of cell clones obtained in step S1 is 195, 200, 205, 215, 220 or 225. In some embodiments, the number of cell clones obtained in step S1 is 195-215.

In some embodiments, the method further comprises step S0: resuscitating frozen HEK293 cells and subculture the cells for more than three generations. Optionally, step S0 comprises: resuscitating frozen HEK293 cells with VP-SFM or Pro293 medium containing 1% (v/v) FBS and 3.5-4.5 mM GlutaMax™.

S2. Evaluating growth rate and transfection efficiency of the cell clones obtained after being transfected with viral vectors, and selecting 15-25 groups of cell clones to be expanded.

In some embodiments, transfection agent used in step S2 is PEIpro, PEI MAX or VirusGen. In some embodiments, the ratio of transfection reagent to viral vector is (1-5):1, and the vector has a concentration of 0.5-2 μg/cm². In some embodiments, the viral vector is AAV transfer plasmid or lentivirus transfer plasmid. It can be understood that, in other embodiments, the viral vector is not limited to AAV or lentivirus, but also other vectors, such as ADV, OV, etc. In some embodiments, the growth rate is evaluated using amplification multiples. It can be understood that in other embodiments, the method of evaluating the growth rate is not limited to the above, but can also be other methods. It can be understood that cell clones to be selected are cell clones with faster growth rate and better transfection efficiency.

S3. Expanding the 15-25 groups of cell clones respectively, and selecting 5-13 groups of optimal cell clones based on growth rate and transfection efficiency during expansion culture.

In some embodiments, the methods for evaluating growth rate and transfection efficiency are as described in step S2. Furthermore, 25 cloned cells with highest growth rate and transfection efficiency of more than 75% are selected for suspension domestication. In some embodiments, 25 cloned cells with highest growth rate and transfection efficiency of 70-95%, such as more than 80%, are selected for suspension domestication.

In some embodiments, the cell clones are arranged according to the transfection efficiency, and the 15-25 groups of cell clones with highest transfection efficiency are selected for suspension domestication. In one embodiment, 15, 18, 20, 22 or 25 groups of cell lines with highest transfection efficiency are selected for suspension domestication. In some embodiments, six-well plates, T25 bottles, T75 bottles, T175 bottles and T225 bottles are used to expand the culture successively.

S4. Subjecting the selected clone cells of each group to suspension domestication respectively.

In some embodiments, suspension domestication include following operations: adjusting the cell density of each group of preferred clone cells to 0.8-1×10⁶ cells/mL, followed by further oscillatory culture respectively, in which the amplitude can be 25-50 mm, and the rotational speed can be 100-150 rpm; exchanging the medium with fresh (or unused) serum free medium when the cell density is lower than 1×10⁶ cells/mL or cell viability is lower than 85%. Furthermore, in the process of suspension domestication, the cell density is 0.5-6×10⁶ cells/mL.

S5. Taking part of cells for each group of cell clones obtained in S4, and selecting cell clones with high viral vector transfection efficiency therefrom to prepare HEK293 suspension cell lines suitable for production of viral vector.

In some embodiments, transfection is conducted when the cell viability is not less than 90%, for example 90%, 95%, or 98%. In some embodiments, the transfection is conducted when the cell viability is not less than 95%.

In some embodiments, transfection agent used in S5 is PEIpro, PEI MAX or VirusGEN. In some embodiments, the ratio of transfection agent to viral vector is (1-5):1, and the viral vector has a concentration of 1-4 μg/mL. In some embodiments, the cells have a density of 2-4×10⁶ cells/mL when transfected.

In some embodiments, the transfection agent is PEIpro, the ratio of transfection agent to AAV transfer plasmid is 2:1, the AAV transfer plasmid has a concentration of 1-2 g/mL, and the cells have a density of 2-4×10⁶ cells/mL when transfected.

In some embodiments, cells with transfection efficiency of more than 85% are selected as HEK293 cells suitable for suspension culture. Furthermore, HEK293 cells with transfection efficiency of more than 90% are selected for suspension culture.

The development method of the application can achieve the suspension domestication of adherent cells in 1-5 weeks, greatly shortening the suspension domestication cycle. During suspension domestication, no additional complex large-scale cloning screening equipment (such as ClonePix, Nanocell) or complex cloning screening technology (such as AI) are required. In addition, the development method of the invention can obtain a cell line with high density and high transfection efficiency, and the cell line can grow in a domestic serum free medium (such as a domestic medium limited by chemical components without animal origin components or protein components) with high density, which not only ensures the safety of biological medicine production, but also greatly reduces the manufacturing cost of biological medicine and the procurement cycle of biological materials. Finally, during suspension domestication with the method of present invention, bioreactor can be used for linear scale-up production, so as to realize the large-scale production of biomedicine (including AAV virus vector, LV virus vector, oncolytic virus, adenovirus vector, vaccines and exosomes based on adenovirus vector).

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention. Unless specified, the adherent cell culture in the following examples was carried out at 37° C. and 5% CO₂, and the suspension domestication was carried out at 37° C. and 8% CO₂.

### Example 1

This example illustrates a method for suspension domestication of an HEK293T cell suitable for lentivirus production. The method comprises the following steps:

S1. Screening cell clones suitable for lentivirus packaging from adherent HEK293T cells, which specifically includes the following sub-steps:

A1. Resuscitating the HEK293T cells growing in adherent cell culture, and culturing the cells adherently using DEME medium containing 10% FBS.

A2. Conducted adherent culture using VP-SFM complemented with 2 mM GlutMax and 10% FB after the cells in step A1 were in well-growing state.

A3. Plating the cells in step A2 into a 96-well cell culture plate using limiting-dilution method at 5-20 cells/mL, preferably 10 cells/mL, and adding 50-100 μL, such as 100 μL, of diluted cultural fluid to each well, then adding 50-100 μL, such as 100 μL, of complete medium containing VP-SFM and 10% FBS to each well.

A4. After culturing for 5-7 days, exchanging part of complete medium comprising VP-SFM medium and 10% FBS, preferably half of the complete medium comprising VP-SFM medium and 10% FBS. Continuing the culture until the cell clone was expanded to the required amount. Then the required amount of cell clone was digested with trypsin, which is usually carried out when the cell clone is expanded to ⅔ of the bottom of the 96-well. Some cells in the cell clone were further cultured as seed cells, usually half of the cells, and the remaining cells were transfected with lentivirus transfer plasmid carrying EGFP gene in another new 96-well plate, and these cells were labeled. Transfection was conducted using EGFP lentivirus transfer plasmid after culturing for 24 hours. During transfection, the plasmid had a concentration of 0.1-0.6 μg/cm², such as 0.4 μg/cm². The V/W ratio of transfection reagent PEIpro to plasmid was 1-3:1, such as 2:1. After 48 hours, green fluorescence was observed using a fluorescence microscope, and the cloned cells with high green fluorescence luminescence points were expanded. Usually, 10 to 100 (preferably 50) cloned cells with the highest fluorescence can be selected for expansion culture, and the cells were cryopreserved for backup during the expansion culture. FIG. 2A and FIG. 2B show the fluorescence images of cloned cells with low transfection efficiency and cloned cells with high transfection efficiency when observing the transfection with EGFP lentiviral transfer plasmid using a fluorescence microscope, respectively. As can be seen from FIG. 2A and FIG. 2B, the brightness of cloned cells with high transfection efficiency is much higher than that of cloned cells with low transfection efficiency, therefore it is easy to screen out cloned cells with high transfection efficiency.

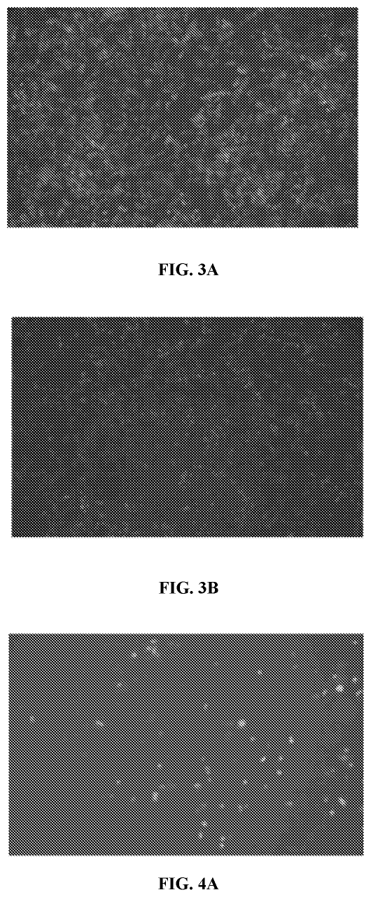

A5. Plating the cloned cells expanded in step A4 into a 24-well cell culture plate at a plating density of 5-9E+04 cells/cm², such as 8E+04 cells/cm². After culturing for 24 hours, EGFP lentiviral packaging was carried out using the lentiviral packaging plasmid with the following conditions: the plasmid had a concentration of 0.1-0.6 μg/cm², such as 0.4 g/cm², and the DNA molar ratio of four plasmids virus transfer plasmid comprising EGFP gene, Pol/gag, VSV-g and Rev was 2:1:1:1, the V/W ratio of transfection reagent PEIpro to plasmid DNA was 1-3:1, such as 2:1. The supernatant of lentivirus carrying EGFP was harvested at 48 h post transfection, and was used to infect the plated HEK293T cells at different dilution times. The plating density of HEK293T cells can be 5-9E+04 cells/cm² such as 8 E+04 cell/cm², and the medium of HEK293T cells plated was DMEM comprising 10% FBS. The transfected HEK293T cells were collected after 48-72 hours, and the infection efficiency was detected by fluorescence microscope and FACS to determine the first few cloned cells with higher transfection efficiency. The resulted cloned cells with high transfection efficiency were expanded and built into a bank. Usually, 10 to 50 cell clones with the highest transfection efficiency can be selected for expansion culture in complete medium containing SFM medium and 10% FBS. After the cells were in well growing state, adaptive culture was conducted using complete medium containing VP-SFM, 2 mM GlutMax and 1% FBS to prepare for suspension domestication. FIGS. 3A and 3B show the results of low-titer EGFP lentivirus infection and high-titer EGFP lentivirus infection in the transduced cells observed by fluorescence microscopy respectively. It can be seen from FIG. 3A and FIG. 3B that the expression of EGFP in the cells transduced with virus produced by high-titer packaging cloned cells is much higher than that of low-titer packaging cloned cells, so it is easy to screen out high-titer packaging cloned cells.

S2. Suspension domestication of the screened HEK293T adherent cells, specifically including the following steps:

B1. The 10 cloned cells with the highest transfection efficiency in step A5 were cultured in complete medium containing VP-SFM, 2 mM GlutMax and 1% FBS to well growing, and then expanded with FBS-free VP-SFM medium. The resulted cells were collected for determination of viable cell density.

Resuspend the cells at a cell density of 5-9E+05 cells/mL using the chemically defined suspension medium containing Transpro CD01, 2 mM GlutMax and 0.1% Anti-clumping agent, such as 8 E+05 cells/mL cells Density resuspend the cells, then use a cell culture shaker for shaking culture at a rate of 125 rpm, and then carry out adaptive subculture at a cell density of 5-8E+05 cells/mL until the cell viability rate reaches 95%.

S3. Screening of high-titer lentiviral packaging clone cells from suspended HEK293T cells. The cells with a viability rate of 95% were inoculated into a 24-well plate using a medium comprising TransproCD01, 4 mM GlutaMax™ and 0.1% Anti-clumping agent and subjected to testing for packaging lentivirus carrying EGFP. When the density of the lentiviral packaging cells was 2E+06 cells/mL, EGFP lentiviral package was carried out with the following conditions: the plasmid had a concentration of 1-3 μg/mL, for example, the four plasmids (EGFP transfer plasmid, Pol/gag, VSV-g and Rev) had a concentration of 2 g/mL, the DNA molar ratio of four plasmids was 2:1:1:1, the V/W ratio of transfection reagent PEIpro to plasmid DNA was 1-3:1, preferably 2:1. The supernatant of lentivirus comprising EGFP gene was harvested at 48 h post transfection and was used to transduce HEK293T cells plated at different dilution times. After 48-72 hours, the infected HEK293T cells were collected, and the infection efficiency was detected by fluorescence microscope and FACS, so as to determine the cloned cells with the highest virus production efficiency. The identified cell clones with the highest virus production efficiency were expanded and used to build a bank to obtain domesticated HEK293T cells suitable for lentivirus production through suspension culture, which were named HEK293TG2S.

According to the above examples, it can be seen that the method for suspension domestication of HEK293T of the present invention can shorten the domestication time from at least 1 month to 7-14 days, and the amount of medium used during the domestication process can also be greatly reduced, reducing the production costs.

### Example 2

This example illustrates the conditions and mediums used for resuspension and subculture of the cell clones.

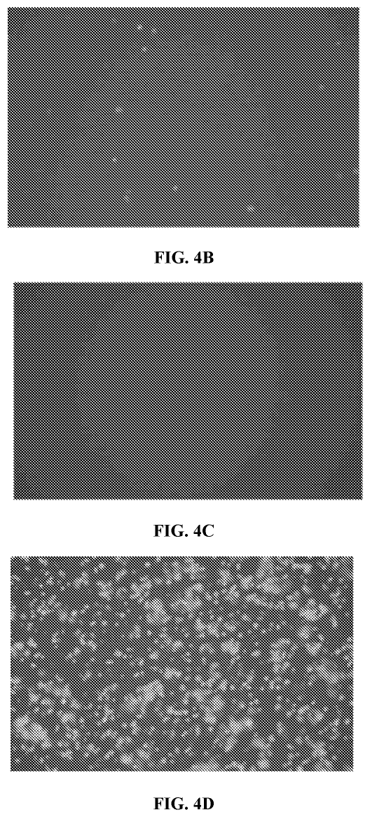

FIG. 4 show the fluorescence images of cells transfected with EGFP transfer plasmid after resuspended and subculture with Dynamis medium, LV-MAX medium, SFM4HEK293, and Transpro CD01 medium in step B2. It can be seen from FIG. 4 that when the medium used for resuspension and subculture was Transpro CD01 medium, the cells showed the highest fluorescence brightness after transfection with EGFP transfer plasmid, which represents the highest transfection efficiency, thus the obtained cells produced highest titers of virus after lentivirus packaging.

### Example 3

This example demonstrates the production of CAR-T cells using the lentivirus produced by the HEK293TG2S generated in Example 1.

Resuscitated the HEK293TG2S generated in Example 1, used Transpro CD01 comprising 0.1% Anti-clumping agent for suspension shaking culture, diluted the well-growing cells at 1E+06 cells/mL, and continued shaking culture for 24 hours, then conduct virus packaging.

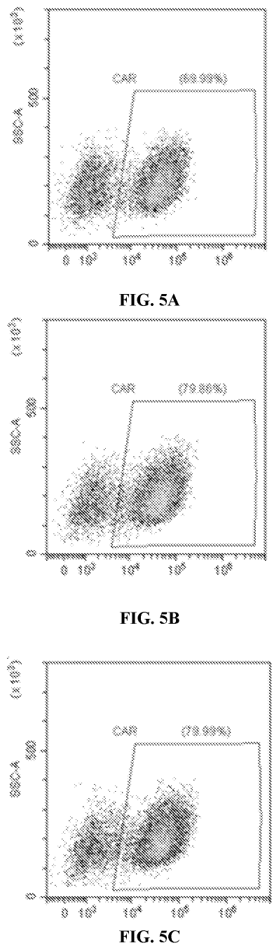

The conditions for virus packaging were as follows: lentiviral packaging cell density was 2E+06 cells/mL, plasmid concentration was 2 μg/mL, the DNA molar ratio of four plasmids (transfer plasmid, Pol/gag, VSV-g and Rev) was 2:1:1:1, the V/W ratio of transfection reagent PEIpro to Plasmid DNA was 2:1. Collect the cell culture supernatant at 48 hours post transfection and measure the infection titer (through FACS) and HCP. The infection titer was above 1.28E+07 TU/mL (transduction units per milliliter) as measured by using the fluorescence labeled target antigen of CAR, and the HCP was 2.7 μg/mL. Using this condition for 1 L-scale lentivirus preparation, the purified virus titer was 1.29E+08 TU/mL and HCP was 722.1 ng/mL. As shown in FIG. 5, when activated T cells were transduced with purified virus at MOI (multiplicity of infection) of 1.3, 3.9, and 6.5, respectively, the corresponding CAR positive cells were 69.99%, 79.86%, and 79.99%.

It can be seen from this example that the viral supernatant obtained after plasmid packaging using the suspension-domesticated HEK293TG2S cells of the present invention has a high infection titer, which can reach more than 1.28E+07 TU/mL, and the HCP content in the virus supernatant is very low, at 2.7 μg/mL. Compared with the existing similar products, the present invention greatly reduces the HCP content and ensures a high virus titer. After purification, the positive rate of T cells transduced and activated is very high, which can significantly enhance the therapeutic effect of CAR.

### Example 4

This example demonstrates the production of CAR-T cells using the lentivirus produced by the HEK293TG2S generated in Example 1.

Resuscitated the HEK293TG2S cells domesticated by suspension in the above example, used Transpro CD01 comprising 0.1% Anti-clumping agent for suspension shaking culture, diluted the well-growing cells at 1E+06 cells/mL, and continued shaking culture for 24 hours, then conducted virus packaging.

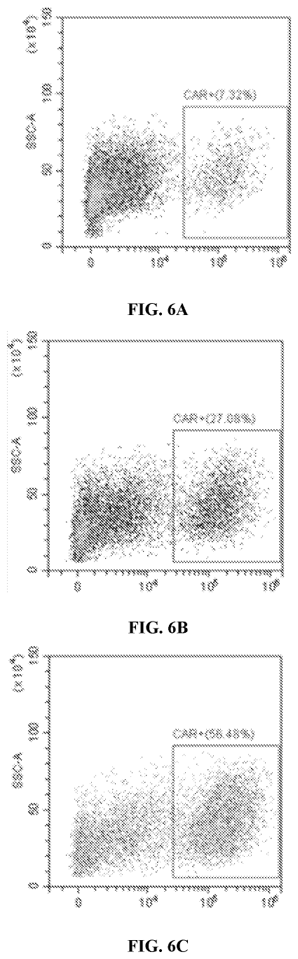

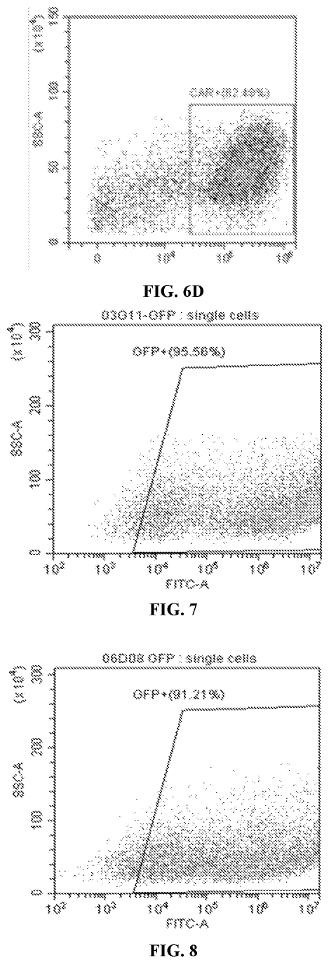

The conditions for virus packaging were as follows: lentiviral packaging cell density was 2E+06 cells/mL, plasmid concentration was 2 μg/mL, the DNA molar ratio of four plasmids (transfer plasmid, Pol/gag, VSV-g and Rev) was 3:1:1:1, and the V/W ratio of transfection reagent PEIpro to Plasmid DNA was 2:1. Collect the cell culture supernatant at 48 hours post transfection and measure the infection titer (through FACS) and HCP, the infection titer was above 1.08E+08 TU/mL (measured by using the fluorescence labeled target antigen of CAR), and the HCP was 7.6 μg/mL. As shown in FIG. 6, using purified virus to transduce activated T cells with MOI of 1, 3, 5 and 10, the corresponding CAR positive rates were 7.32%, 27.08%, 56.48% and 82.48%, respectively.

It can be seen from this application example that the viral supernatant obtained after plasmid packaging using the suspension-domesticated HEK293TG2S cells of the present invention has a high infection titer, which can reach more than 1.08E+08 TU/mL, and the HCP content is low, such as 7.6 μg/mL. With the increase of multiplicity of infection, the positive rate of CAR increased rapidly, which could significantly enhance the therapeutic effect of CAR.

### Example 5

This example illustrates a method for suspension domestication of an HEK293 cell suitable for AAV production.

### Screening of Adherent Clones

Step 1: Recover cryopreserved HEK293 cells (ATCC NO. CRL1573) using VP-SFM medium (Thermo) supplemented with 1% FBS (Thermo) and 4 mM GlutaMax (Thermo).

Step 2: After more than 3 consecutive passages, use the limiting dilution method to plate 96-well cell culture plates with 1 cell/well, and a total of 1920 wells were plated. The cell culture medium comprised VP-SFM medium, 10% FBS and 4 mM GlutaMax, at 37° C., 5% CO₂ incubator for adherent culture. After culturing for 7 days, the medium was changed at half the volume of the medium and observed. A total of 204 clones were grown in the 96-well cell culture plate. The wells of the monoclonal colonies were numbered and marked, and the medium was changed every three days.

Step 3: By evaluating the transfection efficiency and cell growth rate (according to the amplification fold, see Step 4 below for specific steps) of the 204 clones obtained in Step 2, a total of 22 clones were screened. In this step, the AAV transfer plasmid carrying EGFP was used for transfection with PEIpro. The specific steps included: inoculation with 5×10⁴ cells/cm², and transfection after 24 hours. The plasmid was pAAV-EGFP, and the dosage was 0.5 μg/cm², the transfection reagent was PEIpro (Polyplus), and the ratio of transfection reagent/plasmid dosage was 2:1. 48 h after transfection, a flow cytometer (CytoFlex S, Beckman) was used to detect the transfection efficiency.

Step 4: The 22 clones obtained in Step 3 were gradually expanded and cultured in six-well plates, T25 flasks, T75 flasks, T175 flasks, and T225 flasks. A total of 11 cloned cells were screened (which were frozen stored) by evaluating the transfection efficiency and cell growth rate. The top 5 of them were subjected to the next suspension domestication. The evaluation of transfection efficiency and cell growth rate was conducted as follows.

The method for evaluating the cell growth rate (expansion fold): inoculate a six-well plate or T25, T75 at 5×10⁴ cells/cm², when the cell confluence reaches 80% or after culturing for 72 h, trypsinize the cells, count cells with CountStar Rigel S2 cell counter, and the fold expansion was obtained by dividing the total number of cells by the total number of inoculated cells.

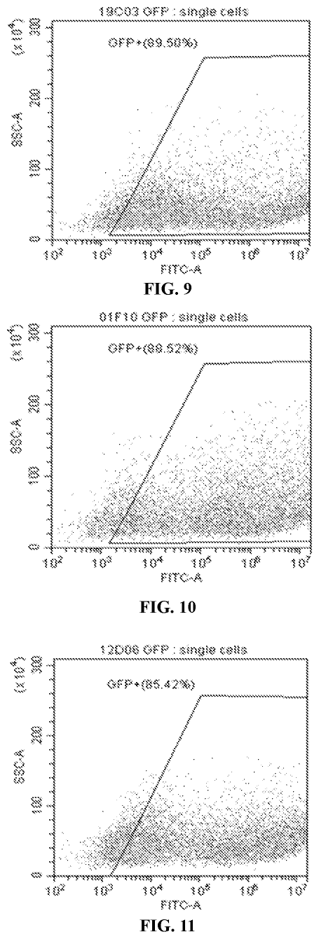

The method of evaluating transfection efficiency: From the adherent cells cultured in 96-well plates, digested the cells with trypsin when the cells reached a confluence of about 80% and amplified the cells in 48-well plates, 24-well plates, 6-well plates, T25 flasks, and T75 flasks consecutively by inoculating the cells at 5×10⁴ cells/cm². Transfected the cells after 24 hours using the plasmid of pAAV-EGFP at the dosage of 0.5 μg/cm² and the transfection reagent PEIpro (Polyplus) at the ratio of transfection reagent/plasmid at 2:1. The transfection efficiency was detected by flow cytometry (CytoFelx S, Beckman) 48 h after transfection. Part of the results of the evaluation of transfection efficiency and cell growth rate are shown in FIGS. 7 to 11, and Tables 1 and 2.

TABLE 1
Ranking of transfection efficiency (pAAV-
EGFP) of the cells in adherent culture

| Clone # | Rank | Transfection efficiency |
|---|---|---|
| 03G11 | 1 | 95.56% |
| 06D08 | 3 | 91.21% |
| 19C03 | 2 | 89.50% |
| 01F10 | 4 | 88.52% |
| 12D06 | 5 | 85.42% |

TABLE 2
Ranking of adherent cell growth (Amplification fold)

| Clone # | Amplification fold | Rank |
|---|---|---|
| 06D08 | 3.31 | 1 |
| 12D06 | 3.09 | 2 |
| 19C03 | 2.89 | 3 |
| 01F10 | 1.86 | 4 |
| 03G11 | 1.69 | 5 |

### Suspension Domestication

Suspension domestication of 5 high transfection efficiency clones screened by adherent culture: before suspension domestication, use complete medium of VP-SFM containing 1% FBS and 4 mM GlutaMax™ to expand the cells with T75, T175, T225 flasks. In the last generation of cell culture before the suspension domestication, cultured the cells using VP-SFM containing 4 mM GlutaMax. The adherent expanded cells were collected, and the cell density was adjusted to 8×10⁵ cells/mL using serum-free medium of SFM4 HEK293 (Cytiva) containing 4 mM GlutaMax and 0.1% (v/v) Anti-clumping agent (Thermo), and seeded at 125 mL cell culture shake flask with an inoculation volume of 20 mL. Then use a cell culture shaker for shaking culture, the speed was 125 rpm (25 mm), the temperature was 37° C., and the CO₂ concentration was set to 8%. Subsequently, samples were taken every 3 to 4 days for live cell counting (ViCell XR, Beckman), and the medium were changed in the culture. This operation was repeated until the cell viability was maintained above 90-95%, the cells were at single cell suspension, and the cell density can reach more than 2×10⁶ cells/mL after culturing for 3-4 days. Then the cells were amplified in 250 mL cell culture shake flask, 500 mL cell culture shake flask and 1000 mL cell culture shake flask and stored by cryopreservation.

### Screening Clonal Cell for Virus Production in Suspension Culture

The use of medium containing SFM4 HEK293 (Cytiva), 4 mM GlutaMax and 0.1% Anti-clumping agent (Thermo) can complete the suspension domestication of adherent cell clones, but due to the low cell growth density and considering supply chain problems, in the subsequent cell culture the following medium were used for the experiment (a total of 11 mediums were tested for the cloned cells obtained in step 1: SFM4 HEK293, SFM4 Transfx293, HEK293 MaxX, OPM 293 CD05, OptiVitro 293, HEK293-04 Prototype, HEK293-13 Prototype, HEK Vip NX, HEK Vip NB, HEK TF, HEK GM). The 5 suspension cloned cells (screened in step 1) were recovered and cultured with the medium OPM 293 CD05 (Shanghai Opmax Bio) and HEK293 MaxX (Shanghai Max Bio), supplemented with 4 mM GlutaMax (Thermo Bio). Due to the removal of the anti-clumping agent, some cells may aggregate during cell culture. After 4 days of cell culture, the cells in the shake flask were transferred to 50 mL centrifuge tubes, and aliquoted into 40 mL tubes at room temperature. After standing for 10 min, a sterile pipette was used to take half of the cell suspension from the liquid surface to a new centrifuge tube, and 0.6 mL of the cell suspension was taken to count live cells using a cell counter (ViCell XR, Beckman). According to the viable cell density, complete medium was used to adjust the cell seeding density to 8-10⁶ 10⁵ cells/mL, and the cell treatment and expansion culture was repeated until the cell viability rate was maintained above 92-95%, the viable cell density was more than 3×10⁶ cells/mL, and the cells were not obviously clumped. The conditions for suspension culture was as follows: the rotation speed was 140 rpm (25 mm), the temperature was 37° C., and the CO₂ concentration was set to 8%.

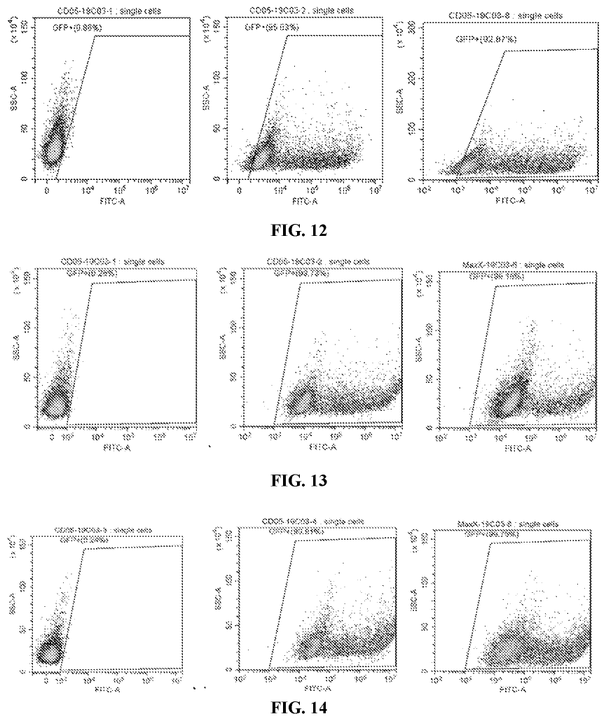

Evaluation of transfection efficiency: OPM 293 CD05 and HEK293 MaxX were used to inoculate the cells at 8×10⁵ cells/mL. After 3 days of shaking culture, the cells were diluted to 3×10⁶ cells/mL with complete medium and transfected with AAV transfer plasmid (pAAV-EGFP) and LV transfer plasmid (LV-EGFP) after 24 h. 48 h after transfection, the transfection efficiency was detected by flow cytometry (CytoFelx S, Beckman). The results are shown in FIGS. 12 to 14 and shown in Table 3. In Table 3, CD05 refers to medium OPM HEK293 CD05, MaxX refers to medium HEK293 MaxX; LV-EGFP and pAAV-EGFP transfection conditions: cell transfection density 4×10⁶ cells/mL, plasmid dosage 2 μg/mL, PEIpro 4 μL/mL, VirusGEN 6 μL/mL; NA means not determined.

TABLE 3
Summary of transfection efficiency tested by 5 suspension
cell clones, 2 media and 2 transfection reagents

| | LV-EGFP | | | | pAAV-EGFP | | | |
|---|---|---|---|---|---|---|---|---|
| | PEIpro | | VirusGEN | | PEIpro | | VirusGEN | |
| Clone # | CD05 | MaxX | CD05 | MaxX | CD05 | MaxX | CD05 | MaxX |
| 19C03 | 95.03% | 61.17% | 92.87% | 79.39% | 99.73% | 99.18% | 99.91% | 99.75% |
| 12D06 | 28.07% | 22.62% | 83.62% | 94.46% | NA | NA | NA | NA |
| 01F10 | 6.72% | 28.61% | 48.13% | 96.66% | NA | NA | NA | NA |
| 03G11 | 7.11% | 2.39% | 36.05% | 80.84% | 18.03% | 11.37% | 97.90% | 99.45% |
| 06D08 | 1.50% | 4.97% | 17.74% | 29.00% | NA | NA | NA | NA |

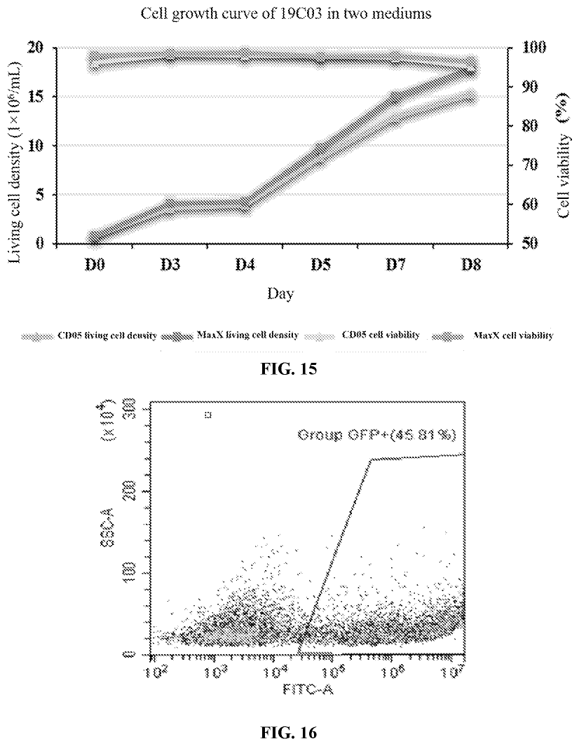

To evaluate the cell growth curve, OPM 293 CD05 and HEK293 MaxX were used to inoculate cells (clone number: 19C03) at 8×10⁵ cells/mL in 250 mL shake flask, and the inoculation volume was 60 mL. Then, the samples were taken at different time points for live cell counting to draw the cell growth curve. The results are shown in FIG. 15.

### Example 6

This example illustrates the comparison of the cell line generated in Example 5 with the HEK293 cells without clone selection and suspension domestication.

The resuscitated and frozen HEK293 cells were transfected with the AAV transfer plasmid carrying EGFP and PEIpro. The specific steps included: inoculation with 5×10⁴ cells/cm², and transfection after 24 hours. The plasmid is pAAV-EGFP, and the dosage is 0.5 g/cm², the transfection reagent was PEIpro (Polyplus), and the ratio of transfection reagent/plasmid dosage was 2:1. 48 hours after transfection, a flow cytometer (CytoFelx S, Beckman) was used to detect the transfection efficiency. The results are as follows shown in FIG. 10. It can be seen from FIG. 16 that the transfection efficiency of HEK293 cells without clone selection and suspension domestication is only 45.81%. Therefore, compared with HEK293 cells without clone selection and suspension domestication, the transfection efficiency of HEK293 cells after clone selection and suspension domestication was greatly improved.

Although embodiments of the present invention have been shown and described, it will be understood by those skilled in the art that various changes, modifications, and substitutions can be made in these embodiments without departing from the principle and spirit of the invention and modifications, the scope of the present invention is defined by the appended claims and their equivalents.

What is claimed is:

1. A method for generating a cell line for producing virus in suspension cell culture, the method comprising:
(i) generating a plurality of cell clones growing in adherent cell culture, each cell clone comprising a group of cells sharing a common ancestry cell;
(ii) identifying a group of cell clones with high transfection efficiency by:
allocating a cell fraction from each cell clones, respectively;
transfecting the cell fraction from each cell clones, respectively, with a report plasmid comprising a first reporter gene; and
identifying a group of cell clones with high transfection efficiency from the plurality of cell clones according to the expression of the first reporter gene in the cell fraction from each cell clones;
(iii) generating a plurality of cell clones growing in suspension cell culture by:
allocating a cell fraction from each of the group of cell clones with high transfection efficiency, respectively;
culturing the cell fraction from each of the group of cell clones with high transfection efficiency, respectively, in a medium that allows the cell fraction from each of the group of cell clones with high transfection efficiency to grow in suspension cell culture, thereby generating a plurality of cell clones growing in suspension cell culture; and
(iv) generating a cell line suitable for producing virus in suspension cell culture by:
allocating a cell fraction from each of the plurality of cell clones growing in suspension cell culture, respectively;
transfecting the cell fraction from each of the plurality of cell clones growing in suspension cell culture, respectively, with (a) a virus transfer plasmid comprising a second reporter gene and (b) a virus packaging plasmid;
collecting virus produced by the cell fraction from each of the plurality of cell clones growing in suspension cell culture, respectively;
transducing a cell with the virus produced by the cell fraction from each of the plurality of cell clones growing in suspension cell culture, respectively; and
selecting a cell clone producing high titers of virus from the plurality of cell clones growing in suspension cell culture according to the expression of the second reporter gene in the cell, thereby generating the cell line suitable for producing virus in suspension cell culture.

2. The method of claim 1, wherein the first reporter gene and/or the second reporter gene encode(s) a fluorescent protein.

3. The method of claim 1, wherein in step (ii), the cell fraction from each cell clones is transfected after logarithmic growth phase.

4. The method of claim 1, wherein in step (iii), the medium is a serum free medium.

5. The method of claim 4, wherein the serum free medium contains VP-SFM, SFM4 HEK293, SFM4 Transfx293, HEK293 MaxX, OPM 293 CD05, OptiVitro 293, HEK293-04 Prototype, HEK293-13 Prototype, HEK Vip NX, HEK Vip NB, HEK TF or HEK GM medium

6. The method of claim 4, wherein the serum free medium contains 2-8 mM L-glutamine or L-alanyl-L-glutamine dipeptide.

7. The method of claim 4, wherein the cell fraction from each of the group of cell clones is cultured in an expansion culture medium before being culture in the serum free medium.

8. The method of claim 7, wherein the expansion culture medium contains (a) VP-SFM medium, or (b) Pro293 medium and 2% (v/v) FBS.

9. The method of claim 8, wherein the expansion culture medium further contains 2-8 mM L-glutamine or L-alanyl-L-glutamine dipeptide.

10. The method of claim 4, when culturing the cell fraction from each of the group of cell clones with high transfection efficiency, the cell fraction from each of the group of cell clones has a density of 0.5-6E+06 cells/mL.

11. The method of claim 4, wherein the cell fraction from each of the group of cell clones is cultured in an adaptive subculture medium after being cultured in the serum free medium.

12. The method of claim 11, wherein the cell fraction from each of the group of cell clones is cultured in the adaptive subculture until cell viability is 93-98%.

13. The method of claim 11, wherein the adaptive subculture medium contains Transpro CD01 medium, 4 mM GlutaMax and 0.1% Anti-clumping agent.

14. The method of claim 11, wherein the cell fraction from each of the group of cell clones is cultured in the adaptive subculture medium when the cell fraction has a density of 5-8E+05 cells/mL.

15. The method of claim 1, wherein the expression of the second reporter gene is measured via flow cytometry or fluorescence microscope.

16. The method of claim 1, wherein the cell fraction of step (ii) or (iv) is transfected using PEIpro, PEI MAX or VirusGen.

17. The method of claim 1, wherein the report plasmid has a concentration of 0.1-0.6 g/cm².

18. The method of claim 1, wherein the virus transfer plasmid and/or the second reporter gene has a concentration of 1-5 μg/mL.

19. The method of claim 1, wherein the plurality of cell clones in step (i) is cultured in a medium containing (a) VP-SFM or Pro293 medium, (b) 2-8 mM GlutaMax, and (c) 2-10% (v/v) FBS.

20. The method of claim 1, wherein step (ii) further comprises screening cell clones producing high tier virus in adherent cell culture from the cell clones with high transfection efficiency.
